(19) United States
(12) Patent Application Publication
(10) Pub. No.: US 2022/0306964 A1
(43) Pub. Date: Sep. 29, 2022

(54) SHEAR-INDUCED PHASE INVERSION OF COMPLEX EMULSIONS FOR RECOVERY OF ORGANIC COMPONENTS FROM BIOMASS

(71) Applicant: The University of Melbourne, Melbourne, Victoria (AU)

(72) Inventors: Gregory John Oliver MARTIN, Melbourne, Victoria (AU); Muthupandian ASHOKKUMAR, Melbourne, Victoria (AU); Wu LI, Melbourne, Victoria (AU)

(21) Appl. No.: 17/618,828

(22) PCT Filed: Jun. 12, 2020
(86) PCT No.: PCT/AU2020/050596

§ 371 Date: Dec. 13, 2021

(30) Foreign Application Priority Data
Jun. 14, 2019 (AU) 2019902082

(51) Int. Cl. C11B 1/10; C09B 61/00; B01D 17/04; B01D 17/02; B01D 11/04

(52) U.S. Cl. CPC C11B 1/10; C09B 61/00; B01D 17/048; B01D 17/0217; B01D 11/0492

(57) ABSTRACT

A method of recovering organic components from an aqueous biomass in the method includes: (i) providing an aqueous biomass containing organic components; (ii) treatment of the aqueous biomass to release intracellular organic components from within cells of the biomass to form a biomass suspension; addition of a water-immiscible component to the biomass suspension to form a mixture comprising biomass and water-immiscible component; (iv) subjecting the mixture comprising biomass and water-immiscible component to high shear to form a water-in-water-immiscible-component emulsion; and (v) separating the water-immiscible component phase from the water/aqueous phase.

### TECHNICAL FIELD

The present invention relates to the recovery of organic components of interest from biomass. In particular the present invention relates to a process for the recovery of organic components from aqueous biomass systems. The process is applicable to a wide range of biomass and is particularly applicable to recovery of organic components from algae, plants (or parts thereof), fungi, bacteria, protists and combinations thereof.

### BACKGROUND OF INVENTION

Biomass is used for the production of a wide range of organic components of interest to man. This is due to the wide range of organisms that can be cultivated as a biomass including algae, plants (or parts thereof), fungi, bacteria, protists and combinations thereof. Due to the wide variety of organisms that can be cultivated/cultured to produce biomass, there is a correspondingly wide variety of organic components that can be recovered. Accordingly, biomass can be used in the generation of feedstock for bioenergy in the form of oils or carbohydrates as well as in the production of human food and/or animal/aquaculture feed and as chemical precursors for further elaboration.

Biomass is also used in the production of organic components such as lipids, carbohydrates and proteins that can be used as feed supplements for animals and aquaculture and as nutritional supplements and ingredients for human food.

Biomass can be produced from terrestrial plants, heterotrophic microorganisms, or by photoautotrophic algae. An example of a biomass that is particularly of interest is algal biomass. Algal biomass production is particularly attractive as it can be carried out on a relatively large scale without the need for large tracts of arable land or an organic carbon source, as the algae grow in the presence of suitable amounts of carbon dioxide and sunlight. Furthermore, due to the high productivity of algae, the overall yield from a biomass cultivation/culturing process will typically be higher per unit area of land than terrestrial crops, with harvests that can be carried out year-round.

Algal biomass finds application in the production of a large number of organic components of interest typically lipids, pigments, proteins and carbohydrates with triglyceride lipids (oils) commonly being the desired extractable organic component from an algal biomass. Lipids of this type find application in a wide variety of industries such as biofuel production, food additives, in cosmetics and in healthcare.

Whilst the ability of algae and heterotrophic microorganisms to produce organic components of interest is generally well known, the application of biomass is limited by the extraction and recovery steps. In general, commercial processes for producing products from biomass are energy-intensive, may use toxic solvents and are typically very costly. This limits the application of these processes to the recovery of high-value products.

By way of example, the extraction of lipids from algal biomass typically involves the use of an organic solvent such as hexane to extract the lipid from the biomass. This has a number of disadvantages such as the need to use energy to thermally remove the solvent from the extracted compounds, the process hazards related to flammability, and the toxic nature of the solvent that may render the remainder of the biomass unsuitable for certain applications due to residual solvent in the biomass after recovery of the organic components of interest. For example, this has meant that in the processing of biomass to produce polyunsaturated fatty acids, the solvent extraction process is such that in many instances, the delipidated biomass is discarded to landfill.

In addition, a further complication of the solvent extraction process is that to be effective the biomass must either first be dried, which requires a lot of thermal energy, or alternatively extractions can be performed on wet biomass in which case the process typically involves the formation of a complex oil-in-water type emulsion that is very difficult to effectively break leading to low extraction efficiency. Drying requires more energy than is contained in the extracted lipids. For extractions on wet biomass, it is estimated that approximately 50% of the equipment costs of a solvent extraction process is attributable to the costs associated with high-speed centrifuges required to break the complex emulsion to allow recovery of the organic components of interest. The costs of energy and equipment are high enough to limit the commercial viability of processes of this type to high-value products as discussed above.

Accordingly, it would be desirable to provide a process for the recovery of organic components from biomass which overcomes one or more of these issues. The ability to recover organic components from aqueous biomass, such as algae, in a cost-effective manner is desirable as it allows the expansion of the market for organic components from biomass as they can be recovered at an economically acceptable cost point.

### SUMMARY OF INVENTION

As a result of the desire to provide an alternative process for the recovery of organic components from an aqueous biomass, the applicants have identified a relatively straightforward process that can be utilised in a number of industrial applications and which overcomes a number of the issues identified.

Accordingly, the present invention provides a method of recovering organic components from an aqueous biomass, the method comprising the steps of: (i) providing an aqueous biomass containing organic components; (ii) treatment of the aqueous biomass to release intracellular organic components from within cells of the biomass to form a biomass suspension; (iii) addition of a water-immiscible component to the biomass suspension to form a mixture comprising biomass and water-immiscible component (iv) subjecting the mixture comprising biomass and water-immiscible component to high shear to form a water-in-water-immiscible-component emulsion; and (v) separating the water-immiscible component phase containing the organic components from the water phase.

The applicants have found the process applicable to the recovery of organic components from a wide variety of aqueous biomasses. The process is generally very energy efficient and can be tailored to meet the requirements for the processing of varying biomasses with ease.

### DESCRIPTION OF THE DRAWINGS

FIG. 1. Macroscopic images of hexane-biomass mixtures prepared with water (w) and water-glycerol (g). (A) shows the mixtures after handshaking. (B) shows the ‘g’ mixture 2 s after the commencement of sonication (at 20 kHz and 3.2 W/mL). (C) shows the ‘g’ mixture at the end of sonication for 5 s (at 20 kHz and 3.2 W/mL). (D) shows the sonicated mixtures after low-speed centrifugation (34×g for 1 min).

FIG. 2. Macroscopic images of biomass extracted by hexane (HX), decane (DC) and hexadecane (HXDC) after sonication and (A) without centrifugation (A), and (B) after centrifugation (500×g, 1 min). Optical microscopic images of the subnatant biomass layer using (C) hexane, (D) decane and (E) hexadecane as the solvent. Scale bar: 50 μm.

FIG. 3. Bulk appearance of a water-in-oil (W/O) emulsion formed at an oil-to-aqueous-biomass ratio of 1.5:1.0 (A and C) and an oil-in-water (O/W) emulsion formed at an oil-to-aqueous-biomass ratio of 1.0:1.0 (B and D) before and after centrifugation at 1000×g for 3 min, respectively. Optical microscopic images (i-iv) were taken for the sample fractions indicated with arrows. Scale bars: 50 μm.

FIG. 4. The range of initial oil-to-biomass ratios (v/v) that result in shear-induced phase inversion to produce an oil-continuous emulsion as a function of biomass solids concentration is shown in solid grey. The vertical dashed area represents the oil-to-biomass ratio that is required to produce an oil-continuous emulsion from an oil-biomass mixture that was initially at a lower oil-to-biomass ratio and subjected to high shear resulting in a stable oil-in-water emulsion. The triangles and circles represent tested conditions.

FIG. 5. Macroscopic images of hexane-biomass mixture, (A) under rotor-stator premixing step (biomass pH=12); (B) After 20 s ultrasonication at pH=4.5 (B) and 12 (C).

FIG. 6. Scheme of avocado oil extraction with images. (i) peeled and destoned avocado was diced before blending; (ii) avocado puree/biomass after blending; (iii) upon solvent addition; (iv) right after ultrasonication for 20 s with obvious phase separation; (v) stable hexane-biomass mixture upon rotor-stator agitation; and (vi) marked phase separation difference after applying a low centrifugal force (100×g, 1 min).

### DETAILED DESCRIPTION

In this specification, a number of terms are used that are well known to a skilled addressee. Nevertheless, for the purposes of clarity, a number of terms will be defined.

Throughout the description and the claims of this specification the word “comprise” and variations of the word, such as “comprising” and “comprises” is not intended to exclude other additives, components, integers or steps.

As used herein the term “biomass” refers to a mass of living or dead biological material and includes materials in their natural or native states and materials that have been subjected to processing to produce a semi-processed biomass.

As used herein the term “aqueous biomass” refers to either biomass material containing water or to biomass material in an aqueous environment (i.e. where the biomass per se may contain little or no water but is mixed in with additional water).

As used herein the term “culturing” refers to deliberately promoting the growth and multiplication of cells or organisms by providing suitable conditions for the cell or organism to carry out some or all of its natural biological processes such as reproduction or replication, such that the total amount of biomass increases.

An “emulsion” is a mixture of two or more liquids that are normally immiscible (unmixable or unblendable), with one of the liquids forming the dispersed phase and the other liquid forming the dispersion medium. Two liquids can form a number of different types of emulsions. By way of example, oil and water can form an oil-in-water emulsion where the oil is the dispersed phase and the water is the dispersion medium. Alternatively, they can form a water-in-oil emulsion where water is the dispersed phase and oil is the dispersion medium. Multiple emulsions are also possible such as a water-in-oil-in-water emulsion or an oil-in-water-in-oil emulsion. In circumstances where there are multiple similar phases in the same emulsion (like the two oil phases in an oil-in-water-in-oil emulsion) each phase may contain a different solute.

As used herein the term “miscibility” and derivations thereof such as “miscible” refers to the property or ability of two substances to mix in all proportions, or put another way, their ability to fully dissolve in each other at any concentration.

Accordingly as used herein the term “water-immiscible” means that there are certain proportions of the substance where it does not dissolve in water. For example, butanone (methyl ethyl ketone) is significantly soluble in water but is still classed as water-immiscible as these two solvents are not soluble in each other in all proportions.

As used herein the term “oil” refers to any nonpolar chemical substance that is both hydrophobic and lipophilic, and may include triesters of glycerol and fatty acids. Oils are typically liquids at room temperature.

As used herein the term “recovery” refers to the useful separation of the water-immiscible component of interest from the remainder of the aqueous biomass. The term “recovery” can include both direct separation of the water immiscible component(s) of interest or removal of the water immiscible components of interest into a water immiscible solvent. The water immiscible components of interest may then be isolated using known separation techniques.

As discussed above the present invention relates to an improved method for the recovery of organic components of interest from an aqueous biomass. In general, there are a number of discrete steps that typically occur in biomass production systems that will be discussed in more detail.

Organic Components that are Recoverable from Biomass

There are a number of components that may be recovered from biomass and utilised in an industrial sense. For example, biomass may contain lipids, proteins, carbohydrates or pigments to name just a few. The biomass may also contain other organic components of interest such as compounds that may find application as flavourings, fragrances or pharmaceuticals. In general, the organic component present in a biomass may vary widely depending upon the biomass chosen. Indeed, in the modern era, scientists are now genetically engineering organisms such as algae, plants, fungi and bacteria to produce specific compounds of interest.

One example of a particularly interesting organic component that may be isolated from biomass are lipids that contain fatty acyl chains which may be saturated or unsaturated. Lipids of this type find a variety of applications and may be used in the production of biofuels such as biodiesel or jet fuel. Indeed certain species of algae can be “farmed” that produce higher lipid yields per unit area than terrestrial oil crops, making them an attractive crop for the production of feedstock for the commodity fuel industry.

In addition to their use as feedstocks in the fuel industry, many of the lipids can be further processed to provide a useful source of other hydrocarbons for industrial use. For example, many lipids contain hydrocarbons such as saturated and monounsaturated hydrocarbons of C₁₀, C₁₂, C₁₄, C₁₆, C₁₇, or C₁₈ chain lengths. In addition, the lipids can be used as food and cooking oils as alternatives to traditional vegetable oils.

In addition to the saturated and monounsaturated fatty acids, many biomasses contain significant levels of polyunsaturated fatty acids (PUFAs) which have been identified as having a wide variety of uses such as in nutrition and healthcare. These components have been used in infant and adult food, pharmaceutical compositions, and as nutritional supplements.

A wide variety of PUFAs are known with it being preferred that they are long-chain (e.g. C₁₈, C₂₀ or C₂₂) omega-6 or omega-3 fatty acids. These unsaturated lipids include docosahexaenoic acid (DHA, an omega-3); alpha-linolenic acid (ALA, an omega-3); arachidonic acid (ARA, an omega-6); eicosapentaenoic acid (EPA, an omega-3); and gamma-linolenic acid and dihomo-gamma-linolenic acid (GLA and DGLA, respectively, each an omega-6).

The biomass also contains protein that may be of interest for instance as a dietary source for humans or for animal or aquaculture feed applications. In addition, the biomass may contain specific functional proteins such as enzymes, lectins, phycobiliproteins, bioactive peptides, or antimicrobial agents. These proteins may be present in native strains or expressed by genetically modified organisms.

The biomass may also contain a number of carbohydrates of interest such as starch, cellulose, hemicellulose, galactomannans, pectins, agar, alginates, carrageenan and xanthan gum which may be used as a source of sugars for fermentation to a range of products in ethanol and lactic acid, or as food additives for instance as stabilisers or thickening agents.

In addition to lipids, proteins and carbohydrates, the biomass may also contain a number of organic components that may be used as flavourings, pigments, antioxidants or pharmaceutically active compounds. Examples of organic components of this type include pigments such as carotenoids (e.g. β-carotene, astaxanthin, lutein and zeaxanthin) chlorophylls, phycobiliproteins and polyphenols (e.g. catechins and flavonols).

In principle the method of the present invention can be utilised in the recovery of any organic component from an aqueous biomass containing the organic component. The process has been found to be widely applicable to a wide variety of aqueous biomass and can therefore be used in the recovery of a wide range of organic components depending upon the aqueous biomass chosen.

Biomass Production

The first step in the process of the present invention is the provision of an aqueous biomass containing organic components. As discussed above an aqueous biomass may either be a biomass with a high enough water content or it may be formed by diluting insufficiently wet biomass with water or from dry biomass material being mixed with water. There are a number of ways in which an aqueous biomass of this type can be provided.

For example in one embodiment organic material may be mixed with water to form an aqueous biomass. Thus, for example, foods such as avocado or olives or wastes from the food industry such as orange skins, grape pressings and the like can be mixed with water to produce an aqueous biomass containing the organic material. In one embodiment the aqueous biomass is formed by pulping fruit to form an aqueous biomass. Examples of fruits that can be pulped include apples, pears, oranges, grapefruits, mandarins, lemons, limes, nectarines, apricots, peaches, plums, bananas, mangoes, strawberries, raspberries, blueberries, kiwifruit, passionfruit, watermelons, rockmelons, honeydew melons, olives, grapes, tomatoes and avocadoes. In certain embodiments the aqueous biomass is formed by pulping the entire fruit. In certain embodiments the aqueous biomass only contains a portion of the fruit such as the skin. As will be appreciated depending upon the water content of the fruit it may be necessary to add additional water as discussed above.

It is appreciated, however, that in general biomass is typically generated by cultivation or culturing of an organism, such as a plant crop, cultivated algae or microorganism.

As will be appreciated by a skilled worker in the art there are a large number of ways in which biomass can be produced including by cultivation of a suitable plant (and harvesting plants or parts thereof) or organisms such as an algae, fungi, yeast, bacteria or protist under suitable culturing conditions which in general are well known in the art. In one embodiment the aqueous biomass is an algal biomass. In one embodiment the aqueous biomass is a fungal biomass. In one embodiment the aqueous biomass is a bacterial biomass. In one embodiment the aqueous biomass is a protist biomass.

In embodiments where the biomass is an algal biomass the applicants note that there are a large number of algal species that have been cultivated/cultured to form biomass and a great diversity of algal that have yet to be cultivated or isolated. Algae include both microalgae (microscopic in size) and macroalgae/filamentous algae that are observable without a microscope. Examples of microalgae that may be used include species in genera such as Nannochloropsis, Chlorella, Haematococcus, Dunaliella, Scenedesmus, Isochrysis, Phaeodactylum, Chlamydomonas, Navicula, Porphyridium, Botryococcus and Thraustochytrium. Examples of macroalgae that may be used include Porphyra, Macrocystis, Spirogyra, Ulva, Sargassum, Augophyllum, and Oedogonium. In addition to eukaryotic algae, blue-green algae/cyanobacteria (photosynthetic bacteria) can be used including for example Spirulina, Microcytis, Anabaena, Prochlorococcus, Nostoc and Synechocytis.

The applicants have found that the process of the present invention is applicable to the recovery of organic components from a wide range of biomasses. Nevertheless for completeness we will describe the general procedure for the production of biomass from algae.

In general the cultivation of algal biomass typically involves the culturing of the algae (either freshwater, brine or marine) in a suitable culturing media selected based on the characteristics of the algae. Typically this will comprise of a source of water of the appropriate salinity (e.g. fresh water, brackish water, seawater, or hypersaline water) supplemented with nutrients (e.g. sources of nitrogen, phosphorous, minerals, trace elements and possibly vitamins). The exact media selected will vary on the algae type as would be well appreciated by a skilled worker in the art.

The algal species can be cultivated indoors or outdoors in a wide variety of cultivation systems ranging from large open pond systems such as raceway ponds through to tubular or flat panel photo-bioreactors. The choice of system will in general depend upon the scale of the cultivation facility, the capital costs, the specific requirements of the species to be produced, and the factors relating to the production location and other process variables such as available space and energy requirements.

Cultivation of the algal species in these ways may involve the use of natural sunlight or it may involve subjecting the culture to artificial light to allow indoor cultivation or to intensify or lengthen the period of exposure of the culture system to light to increase production. Cultivation may also be performed under mixotrophic conditions in which both light and an organic carbon source such as glucose, glycerol or acetate are provided to the cultures. Alternatively, some algae can be grown heterotrophically, by providing an organic carbon source but not a source of light.

In general algae are cultured at temperatures in the range of 10° C. to 40° C. although depending on the climate and the algal species chosen it is not unknown for culture temperatures to go below or to exceed this for limited periods. The temperatures under which the biomass is cultured can vary geographically and temporally, particularly for outdoor cultures as is well known in the art. For indoor cultures the temperature can readily be selected and controlled by the skilled worker based on the identity of the algal species chosen.

Once the algae has been cultured for a sufficient period to reach a desired biomass concentration, biomass from all or some of the culture medium is then typically harvested to produce an aqueous biomass of appropriate concentration for further processing. As algae are commonly cultured as dilute liquid suspensions harvesting typically involves an initial concentration step using chemical flocculation, membrane filtration or flotation of the algae followed by a further concentration step for example using centrifugation, drum filtration or filter pressing, to produce a concentrated aqueous biomass.

The process described above produces an aqueous biomass suitable for further processing. The properties of the aqueous biomass may vary depending on the type and nature of the biomass and with the solids content depending on the processing conditions used. Reference to the solids content in relation to the aqueous biomass refers to biomass solids i.e. not including intra- or intercellular water or intercellular salts (or ash content). For instance, for algae growth in saline media, the term ‘ash-free’ dry weight is applicable.

In some embodiments the solids concentration of the aqueous biomass will be in the range of from 0.1 wt % to 90 wt %. In some embodiments the solids concentration of the aqueous biomass will be in the range of from 0.1 wt % to 75 wt %. In some embodiments the solids concentration of the aqueous biomass will be in the range of from 0.1 wt % to 60 wt %. In some embodiments the solids concentration of the aqueous biomass will be in the range of from 1 wt % to 50 wt %. In some embodiments the solids concentration of the aqueous biomass will be in the range of from 2 wt % to 45 wt %. In some embodiments the solids concentration of the aqueous biomass will be in the range of from 5 wt % to 40 wt %. In some embodiments the solids concentration of the aqueous biomass will be in the range of from 10 wt % to 35 wt %. In some embodiments the solids concentration of the aqueous biomass will be in the range of from 15 wt % to 30 wt %.

Biomass Treatment—Cell Lysis

In most instances the organic components of the biomass that are intended to be recovered are contained in the cells of the biomass. The process therefore typically requires the treatment of the biomass to rupture the cell walls in order for the biomass to release intracellular components from within the cells of the biomass.

There are a number of ways well known in the art that have been utilised in order to disrupt cell walls/membranes and allow the intracellular components to be released. For example, the cell walls/membranes can be disrupted by subjection of the aqueous biomass to shear, mechanical pressing, high-pressure homogenisation, microfluidisation, enzymatic or chemical treatment, bead milling, microwave irradiation, ultrasonication, pulsed electric fields or osmotic stressing.

Aqueous biomasses could be produced directly from biomass containing water or by combination of a biomass component with water to form an aqueous biomass such as when fruit or a portion thereof is the biomass cell lysis can be achieved by subjecting the biomass to shear or mechanical pressing. This can be achieved, for example by pureeing the fruit either alone or in combination with water to form a fruit puree.

In one embodiment the aqueous biomass is subjected to high-pressure homogenisation. In one embodiment the aqueous biomass is subjected to microfluidisation. In one embodiment the aqueous biomass is subjected to bead milling. In one embodiment the aqueous biomass is subjected to microwave irradiation. In one embodiment the aqueous biomass is subjected to ultrasonication. In one embodiment the aqueous biomass is subjected to a pulsed electric field. In one embodiment the aqueous biomass is subjected to osmotic stressing. In one embodiment the aqueous biomass is subjected to mechanical pressing. In one embodiment the aqueous biomass is subjected to pureeing.

Following rupture of the cell wall as discussed above, the treated biomass typically forms a biomass suspension (which may be wholly or partially in the form of an emulsion) containing water, liquid organic components and solid organic matter. In circumstances where the biomass suspension is an emulsion, the emulsion is typically a complex emulsion wherein the continuous phase is aqueous and the organic components are the dispersed phase stabilised by the cellular organic matter. This is therefore a complex oil-in-water type emulsion which is very stable and hard to break with the result that the phase separation of the organic phase from the aqueous phase is very energy intensive and typically inefficient using current technology including centrifugation and/or the addition of chemical demulsifiers. The exact physical characteristics of the emulsion will depend upon the aqueous biomass precursor with the applicants identifying that where a biomass is treated that has a lower solids content such as 5 wt % the complex emulsion typically has a low viscosity typically <200 cP (25° C., 1 s⁻¹) whereas with higher solids concentrations in the biomass such as 20 wt % the complex emulsion may have a viscosity possibly >10,000 cP (25° C., 1 s⁻¹). The applicants note that high viscosity of the treated biomass at such high concentrations may lead to high energy costs in the later separation processes that can require coalescence and creaming of the water-immiscible phase in order to recover the desired organic component as well as the added water-immiscible agent.

The applicants have found that depending upon the nature of the biomass it may be desirable to subject the biomass to pH adjustment prior to subjecting the biomass to the remainder of the process. In certain cases the applicants have found that it can be desirable to raise the pH of the aqueous biomass as a means of increasing extraction efficiency and separation efficiency.

In general the pH of the aqueous biomass may be adjusted by the addition of either an acid or base depending upon whether it is desired to decrease or increase the pH of the aqueous biomass. Examples of suitable commercially available acids and bases are well known.

In one embodiment the pH of the aqueous biomass is adjusted to be in the range of 5.0 to 13.0. In one embodiment the pH of the aqueous biomass is adjusted to be in the range of 7.0 to 11.0. In one embodiment the pH of the aqueous biomass is adjusted to be in the range of 8.0 to 10.0. In one embodiment the pH of the aqueous biomass is adjusted to be in the range of 8.6 to 9.0.

In one embodiment the pH of the aqueous biomass is adjusted to be in the range of 5.0 to 7.0. In one embodiment the pH of the aqueous biomass is adjusted to be in the range of 6.0 to 8.0. In one embodiment the pH of the aqueous biomass is adjusted to be in the range of 7.0 to 9.0. In one embodiment the pH of the aqueous biomass is adjusted to be in the range of 8.0 to 10.0. In one embodiment the pH of the aqueous biomass is adjusted to be in the range of 9.0 to 11.0. In one embodiment the pH of the aqueous biomass is adjusted to be in the range of 10.0 to 12.0. In one embodiment the pH of the aqueous biomass is adjusted to be in the range of 11.0 to 13.0.

In one embodiment the pH of the aqueous biomass is about 5.0. In one embodiment the pH of the aqueous biomass is about 5.5. In one embodiment the pH of the aqueous biomass is about 6.0. In one embodiment the pH of the aqueous biomass is about 6.5. In one embodiment the pH of the aqueous biomass is about 7.0. In one embodiment the pH of the aqueous biomass is about 7.5. In one embodiment the pH of the aqueous biomass is about 8.0. In one embodiment the pH of the aqueous biomass is about 8.5. In one embodiment the pH of the aqueous biomass is about 9.0. In one embodiment the pH of the aqueous biomass is about 9.5. In one embodiment the pH of the aqueous biomass is about 10.0. In one embodiment the pH of the aqueous biomass is about 10.5. In one embodiment the pH of the aqueous biomass is about 11.0. In one embodiment the pH of the aqueous biomass is about 11.5. In one embodiment the pH of the aqueous biomass is about 12.0. In one embodiment the pH of the aqueous biomass is about 12.5. In one embodiment the pH of the aqueous biomass is about 13.0.

In certain embodiments the aqueous biomass may be subjected to treatment using enzymes with pH adjustments (if this step is included) to facilitate breakdown of interfacial-active biopolymers, such as proteins and carbohydrates present in the aqueous biomass and hence to weaken the emulsion stability and facilitate release of the organic components from the aqueous biomass continuous phase. Enzyme assisted aqueous extraction techniques of this type are well known in the art and may be used in the process of the present invention.

The applicants have also found that the extraction efficiency and separation efficiency may also be affected by the temperature of the aqueous biomass. Accordingly, in some embodiments the applicants have found it to be desirable to adjust the temperature of the aqueous biomass before subjecting it to the reminder of the process. In one embodiment the temperature of the aqueous biomass is adjusted to be between 20° C. to 30° C. In one embodiment the temperature of the aqueous biomass is adjusted to be between 30° C. to 40° C.

Addition of Water-Immiscible Components

As discussed above, processing of the complex biomass suspension formed after rupture of the cells using existing technology is typically inefficient as the suspension is very stable and hard to break meaning that the extraction of the desired organic component from the biomass into the added water-immiscible agent as well as the subsequent phase separation is energy intensive and inefficient. Accordingly, in order to overcome this issue the applicants have developed a technique to cause phase inversion and thus convert biomass suspension to a water-in-oil emulsion. In order to carry out the phase inversion the applicants have found that it is desirable to add additional amounts of a water-immiscible component to the complex biomass suspension as it facilitates the phase inversion step.

The water-immiscible component may take any number of forms with the identity of the water-immiscible component typically being selected on the basis of the desired end-use application of the organic components to be recovered from the biomass and the cost, availability, and properties of the material. For example where the recovered organic components are intended to be used as a food additive it is advantageous to attempt to use a food-grade water-immiscible component. In principle, any water-immiscible component may be used with a water-immiscible liquid being preferred.

In some embodiments, the water-immiscible component is an oil or a combination of oils. The oil may be an organic oil or a mineral oil. Examples of oils that may be used include C₆-C₁₈ hydrocarbons, triglycerides, natural oils, petroleum-based oils, and silicone oils. In some embodiments the oil is a natural oil selected from the group consisting of almond oil, apricot kernel oil, avocado oil, olive oil, safflower oil, sesame oil, soybean oil, sunflower oil, rapeseed oil, hemp oil, canola oil, cocoa butter, peanut oil, wheat germ oil, and other vegetable oils.

In some embodiments the water-immiscible component is a solvent or a combination of solvents. Examples of suitable solvents that may be used include carbon tetrachloride, chloroform, cyclohexane, 1,2-dichloroethane, dichloromethane, diethyl ether, dimethyl formamide, ethyl acetate, heptane, hexane, methyl-tert-butyl ether, pentane, toluene, and 2,2,4-trimethylpentane or a combination thereof. In one embodiment the water-immiscible component is hexane.

In one particularly preferred embodiment the water-immiscible component is the same type of oil as is being extracted from the biomass. Accordingly, where the biomass is an algal biomass it is preferred that the water-immiscible component is algal oil or derivative thereof (e.g. fatty acid methyl esters). Correspondingly, where the biomass is an avocado it is preferred that the water-immiscible component is avocado oil. This is typically recycled from a later stage of the recovery or conversion process either before or after the high-value organic components have been isolated or processed during a refining step. Such a recycle process avoids potential chemical contamination resulting from the addition of organic solvents and obviates the need to separate the product from the extractant (e.g. by energy-intensive distillation in the case of organic solvents).

The amount of water-immiscible component added/recycled will vary depending upon the oil content of the biomass suspension. For example, where the biomass suspension has a relatively low oil content, a larger amount of water-immiscible component is required to be added than is the case where the biomass suspension has a relatively high oil content.

Nevertheless in general the amount of water-immiscible component added to the biomass suspension is sufficient to form a mixture with a ratio of water-immiscible component:biomass suspension of at least 1.0:1.0 (v/v). Whilst the process will work where the amount of added water-immiscible component is less than this ratio, the recovery of organic components from the biomass typically drops.

In one embodiment the amount of water-immiscible component added to the biomass suspension is sufficient to form a mixture with a ratio of water-immiscible component:biomass suspension of at least 1.5:1.0. In one embodiment the amount of water-immiscible component added to the biomass suspension is sufficient to form a mixture with a ratio of water-immiscible component:biomass suspension of at least 2.0:1.0. In one embodiment the amount of water-immiscible component added to the biomass suspension is sufficient to form a mixture with a ratio of water-immiscible component: biomass suspension of at least 2.5:1.0. In one embodiment the amount of water-immiscible component added to the biomass suspension is sufficient to form a mixture with a ratio of water-immiscible component:biomass suspension of at least 3.0:1.0. Whilst the process will work where the amount of added water-immiscible component is greater than this ratio, the process efficiency decreases as this ratio is increased. In each instance the ratio is on a volume-for-volume basis.

Following addition of the water-immiscible component the resulting mixture is typically gently agitated to ensure mixing of the water-immiscible component with the biomass suspension to form a mixture comprising biomass and water-immiscible component. The mixture comprising biomass and water-immiscible component is typically in the form of a multiphase complex emulsion.

Shear-Induced Phase Inversion

The mixture comprising biomass and water-immiscible component produced as discussed above is then subjected to high shear to induce phase inversion to form a water-in-water-immiscible-component emulsion with droplets of a microscopic scale. The process of shear-induced phase inversion results in the partitioning of the organic components of interest into the added water-immiscible agent. In addition, the resulting inverted emulsion has a high interfacial area relative to the volume being processed, enhancing the efficiency of subsequent extraction of the desired organic component of interest into the added water-immiscible agent. The applicants have observed that the phase inversion has the result of changing the biomass suspension from a highly viscous aqueous continuous phase to a lower viscosity water-immiscible substance continuous phase which greatly improves separation efficiency.

As will be appreciated by a skilled worker in the art where the water-immiscible substance is an oil the phase inversion creates a water-in-oil emulsion. There are a number of high shear techniques that can be utilised in order to carry out this high shear-induced phase inversion.

Examples of techniques for providing high shear that produce the necessary shear forces include high-pressure homogenisation, microfluidisation, hydrodynamic cavitation and ultrasonication.

In one embodiment of the process of the invention in step (iv), the mixture comprising biomass and water-immiscible-component is subjected to high shear by sonication of the mixture comprising biomass and water-immiscible component. The sonication frequency used may vary greatly although it is typically in the range of from 20 kHz to 200 kHz. In one embodiment the sonication is carried out at a frequency of from 20 kHz to 200 kHz. In one embodiment the sonication is carried out at a frequency of from 20 kHz to 150 kHz. In one embodiment the sonication is carried out at a frequency of from 20 kHz to 100 kHz. In one embodiment the sonication is carried out at a frequency of from 20 kHz to 40 kHz.

As will be appreciated by a skilled worker in the field sonication frequency is merely one variable in the sonication process. In general, the threshold energy density is a better way of determining the sonication step.

The threshold energy density (E) may be defined as:

E=(P×t)/V;

Where P=Power input (W), t=time (s) and V (mL) is the processed volume.

In certain embodiments, the sonication is carried out at an energy density of greater than 20 J/mL.

The sonication may be carried out for any period of time necessary to achieve the desired phase inversion. In one embodiment the sonication is carried out for from 1 second to 600 seconds. In one embodiment the sonication is carried out for from 5 seconds to 300 seconds. In one embodiment the sonication is carried out for from 10 seconds to 200 seconds. In one embodiment the sonication is carried out for from 15 seconds to 100 seconds. In one embodiment the sonication is carried out for from 20 second to 50 seconds.

In one embodiment the sonication is carried out for about 5 seconds. In one embodiment the sonication is carried out for about 10 seconds. In one embodiment the sonication is carried out for about 15 seconds. In one embodiment the sonication is carried out for about 20 seconds. In one embodiment the sonication is carried out for about 25 seconds. In one embodiment the sonication is carried out for about 30 seconds. In one embodiment the sonication is carried out for about 35 seconds. In one embodiment the sonication is carried out for about 40 seconds. In one embodiment the sonication is carried out for about 45 seconds. In one embodiment the sonication is carried out for about 50 seconds. In one embodiment the sonication is carried out for about 55 seconds. In one embodiment the sonication is carried out for about 60 seconds.

In one embodiment of the process of the invention in step (iv) the mixture comprising biomass and water-immiscible component is subjected to high shear by the subjection of mixture comprising biomass and water-immiscible component to high-pressure homogenisation.

In one embodiment the high-pressure homogenisation is carried out at a pressure of from 10 MPa to 400 MPa. In one embodiment the high-pressure homogenisation is carried out at a pressure of from 10 MPa to 300 MPa. In one embodiment the high-pressure homogenisation is carried out at a pressure of from 10 MPa to 200 MPa. In one embodiment the high-pressure homogenisation is carried out at a pressure of from 20 MPa to 200 MPa. In one embodiment the high-pressure homogenisation is carried out at a pressure of from 50 MPa to 200 MPa. In one embodiment the high-pressure homogenisation is carried out at a pressure of from 50 MPa to 100 MPa.

In one embodiment the high-pressure homogenisation is carried out at a temperature of from 10° C. to 90° C. In one embodiment the high-pressure homogenisation is carried out at a temperature of from 10° C. to 70° C. In one embodiment the high-pressure homogenisation is carried out at a temperature of from 10° C. to 50° C. In one embodiment the high-pressure homogenisation is carried out at a temperature of from 20° C. to 50° C. In one embodiment the high-pressure homogenisation is carried out at a temperature of from 20° C. to 30° C.

In certain embodiments the high-pressure homogenisation involves multiple passes of the mixture comprising biomass and water-immiscible component through the homogeniser. In one embodiment the mixture comprising biomass and water-immiscible component is passed 6 times through the homogeniser. In one embodiment the mixture comprising biomass and water-immiscible component is passed 5 times through the homogeniser. In one embodiment the mixture comprising biomass and water-immiscible component is passed 4 times through the homogeniser. In one embodiment the mixture comprising biomass and water-immiscible component is passed 3 times through the homogeniser. In one embodiment the mixture comprising biomass and water-immiscible component is passed 2 times through the homogeniser.

In one embodiment the mixture comprising biomass and water-immiscible component is passed a single time through the homogeniser.

The flow rate of the mixture comprising biomass and water-immiscible component through the high-pressure homogeniser will be dependent on a number of variables including the exact parameters and scale of the homogeniser equipment, however, the flow rate is typically in the range of from 10 to 28000 L/h.

Phase Separation

Following phase inversion using high shear as discussed above, the applicants have found that the water-in-water-immiscible-component emulsion that is formed is much less stable than the initial mixture comprising biomass and water-immiscible component. This facilitates the separation of the water (aqueous) phase from the water-immiscible component phase. The separation of the two phases is carried out using conventional techniques known in the art. For example the separation may be carried out using gravitational settling, malaxation or centrifugation. In one embodiment the separation is carried out using gravitational settling. In one embodiment the separation is carried out using malaxation. In one embodiment the separation is carried out using centrifugation. In one embodiment the centrifugation is carried out at a force less than 10,000×g for no more than 10 minutes.

In general the organic components from the biomass tend to partition into the water-immiscible phase and so when the two phases are separated, the organic components are found in the water-immiscible phase. The water-immiscible phase may then be subjected to further refining in circumstances where it is desired to further purify the organic components.

Following phase separation, in one embodiment, a portion of the recovered water-immiscible phase is recycled back to the front of the process (i.e. added to the complex biomass suspension as described in above) to act as the extracting agent, and the remaining portion is recovered from the process and possibly further refined or processed. Further refining/processing could include but is not limited to steps such as degumming, transesterification, hydrogenation and purification. Alternatively, while not seen as being necessary, the separated water-immiscible component could be refined/processed prior to being recycled.

In one embodiment the method further comprises the step of (vi) isolating the organic components from the water-immiscible phase.

The exact process used to recover and further refine the organic components from the water-immiscible phase will vary depending on the nature of the organic components. As will be appreciated by a skilled worker in the field different organic components will be recovered from different biomass sources. Indeed different organic components will be recovered from different algal species. In general there are a number of well-known refining techniques that may be utilised.

For example one well-known technique is the use of liquid-liquid extractions that can typically be used in order to selectively extract certain organic components. In this way a skilled worker can modify the liquids used to extract certain components.

As an alternative there is the possibility of using Solid Phase extraction systems containing a solid phase designed for the extraction in hand. In such systems the crude mixture is added to the column and then various elution solutions are used to selectively strip the individual organic components from the column.

In yet an even further alternative the water-immiscible phase may be subjected to distillation to separate out volatile components.

As will appreciated, in principle any biorefining technique known for the further refining of the components of the water-immiscible phase may be used in order to provide the organic components in the required purity.

The invention will now be illustrated by way of examples; however, the examples are not to be construed as being limitations thereto.

### EXAMPLES

### Example 1: Lipid Recovery from Microalgae Biomass by Shear-Induced Phase Inversion Method

Algal Biomass Cultivation

Nannochloropsis sp. monocultures were grown indoors at 20° C. with a light:dark cycle of 14:10 hours using 15 L carboys. The aeration of bioreactors was conducted by aquarium air pumps at a flow rate of 190 L/h. After a 14-day growth period, the algae cultures were harvested and concentrated by a disc stack centrifuge (Separator OTC 2-02-137, GEA Westfalia, Italy). A typical solids concentration range of the concentrated algal paste was around 28 to 32 wt %, which was determined gravimetrically after over drying at 60° C. for 24 h.

Total Lipid and Non-Polar Lipid Fraction Determination

The content of total extractable lipids for every batch was determined by a modified Bligh and Dyer extraction method as described elsewhere (Bligh, E. G. and W. J. Dyer, A rapid method of total lipid extraction and purification. Canadian journal of biochemistry and physiology, 1959. 37(8): p. 911-917.). In short, a mixture of chloroform/methanol/biomass (1.0:2.0:0.8 v/v/v, where biomass applied has a solid concentration around 9 to 10 wt %) was subjected to agitation followed by gravitational separation of the mixture with the addition of extra chloroform and water. The upper aqueous layer was discarded, and the bottom chloroform layer was obtained, after which fresh solvents were added to reach the extraction ratio mentioned earlier. Cycles of this process were performed until the colour of the biomass turned from green to grey. The collected lipids were fractionated into neutral lipid (NL), phospholipid (PL), and glycolipid (GL) via solid phase extraction (Olmstead, I. L., et al., A quantitative analysis of microalgal lipids for optimization of biodiesel and omega-3 production. Biotechnology and Bioengineering, 2013. 110(8): p. 2096-2104).

Cell Disruption

The fresh concentrated algal paste was diluted to around 25 wt % solids concentration, after which the paste was incubated at 40° C. for 24 h to induce cell weakening. The incubated biomass was ruptured by high-pressure homogenisation (Panda 2K NS1001L, GEA Niro Soavi, Italy) at an applied pressure of 1200 bar for 1 pass. The efficiency of cell disruption was determined by cell counting under an optical microscope. The rupture rate is around 80-90% of the total cell number.

Shear-Induced Phase Inversion Recovery of Algal Lipids

Phase inversion of emulsions can occur catastrophically or transitionally. Catastrophic phase inversion, for example of an oil-in-water (O/W) to a water-in-oil (W/O) emulsion, can occur when the composition of the emulsion is changed so that the ratio of dispersed to continuous phase is altered. Transitional phase inversion can occur if the interfacial properties are altered, for example, by the addition of demulsifiers, changes in temperature, changes in the concentration of interfacial active compounds, or alteration of the viscosity of the phases. In addition, exposure to shear can result in a dynamic phase inversion process (Peña, A. and J.-L. Salager, Effect of stirring energy upon the dynamic inversion hysteresis of emulsions. Colloids and Surfaces A: Physicochemical and Engineering Aspects, 2001. 181(1-3): p. 319-323.). The current phase inversion method can involve all these aspects (altering the ratio of the continuous and dispersed phase, altering the interfacial properties, and application of shear) to achieve the enhanced recovery of organic compounds from biomass. In the following examples, both ultrasonication and high-pressure homogenisation (HPH) were used to create high-shear environments. The results from the two methods were found to be comparable, and the required energy to be a similar order of magnitude.

The total recovery efficiency of the algal lipids was determined on the basis of both the extraction efficiency (proportion of the extractable lipids that was partitioned into the added water-immiscible component (canola oil)) and separation efficiency (the proportion of extracted algal lipids and water-immiscible component (canola oil) that is physically separated from the mixture). Separation efficiency was determined gravimetrically after applying centrifugation to separate the canola oil-algae lipid mixture from the residual aqueous biomass. The extraction efficiency was determined by measuring the concentration of algal lipids in the recovered canola oil-algal lipid mixture. The concentration of algal lipids in the recovered canola oil-algal lipid mixture was determined by measuring ultraviolet absorbance at 670 nm, which was correlated to the chlorophyll-a concentration and used as a proxy for the concentration of extractable algal lipids.

Effect of Co-Solvents in the Aqueous Phase

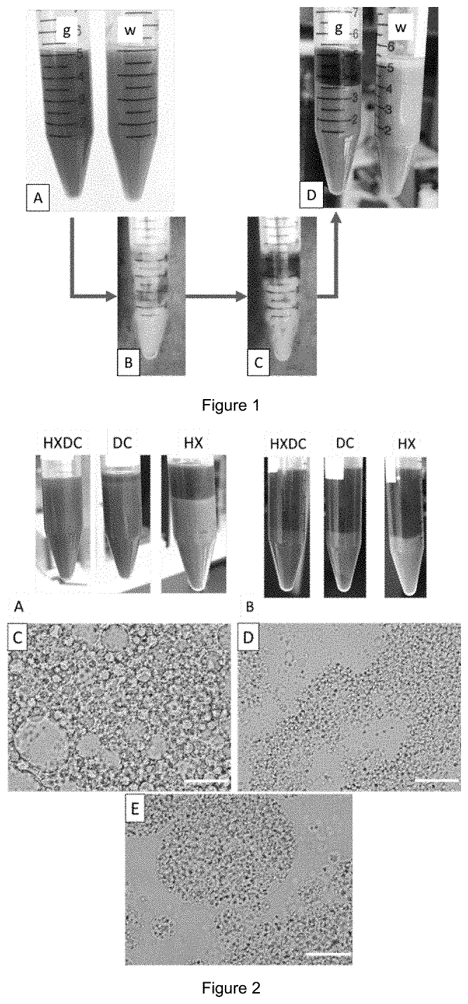

It is generally known that the presence of co-solvents such as polyols in water can alter the interfacial activity macromolecules such as protein and polysaccharides, which stabilise emulsions. Accordingly, transitional phase inversion, for example from an O/W emulsion to a W/O emulsion, can be promoted by changing the formulation. In preliminary tests using a chitosan-based emulsion system, the addition of glycerol as a co-solvent in the aqueous phase reduced the oil fraction required to achieve transitional phase inversion. The effect of glycerol was then examined in an algae biomass system. 10 wt % solids concentration biomass samples were prepared by diluting 20 wt % biomass with water or glycerol. Hexane was used as the solvent and the hexane-biomass ratio was 1.1:1.0 (v/v). As shown in FIG. 1A, both samples were hand-shaken, resulting in the formation of stable O/W emulsions (no phase separation occurred over 24 hours at room temperature). The mixtures were then subjected to ultrasonication at 3.2 W/mL for 5 s. Images taken during and after sonication (FIGS. 1B and C) demonstrate phase inversion from a water-continuous to a hexane-continuous emulsion, as indicated by the dark-green channels of chlorophyll-rich hexane formed around the biomass. Furthermore, the appearance of a clear hexane layer on top on the emulsion could be immediately after sonication (FIG. 10). The pale-green aqueous biomass phase can be seen to sediment naturally under gravity due to the density difference, confirming that the hexane has become the continuous phase. With minimal centrifugal force applied (34×g, 1 min) the hexane was completely separated in the sample with glycerol as a co-solvent. In comparison, the water-diluted sample formed a highly stable gel-like O/W emulsion for this particular composition which had a sub-optimal solvent-to-biomass ratio. The effect of the solvent-to-biomass ratio is explored in Example 9. These results demonstrate that the phase inversion process can be improved by the addition of co-solvents for enhanced oil recovery.

Effect of Carbon Chain Length of Alkanes as the Water-Immiscible Extractants/Solvents

The chemical structure of the water-immiscible component can affect emulsion formation. For example, an increase in the carbon chain length of saturated hydrocarbons will increase the viscosity and hydrophobicity of the fluid. Emulsion formation was tested using three common hydrocarbons: hexane (HX, C₆), decane (DC, C₁₀) and hexadecane (HXDC, C₁₆). 10 wt % biomass was prepared by diluting 20 wt % paste with glycerol. The oil-biomass ratio was maintained at 1.17 on a volumetric rather than mass basis due to the density difference between the hydrocarbons. The same mixing procedure as in example 6 was conducted (hand mixing followed by ultrasonication at 3.2 W/mL for 5 s). The appearance of the emulsions indicates that the samples were oil continuous immediately after sonication (FIG. 2A), with clear phase separation observable in the hexane sample. However, the more viscous water-immiscible phase in the decane and hexadecane samples slowed the phase separation, as indicated by the minor extent of phase separation found in the decane sample and no visible phase separation in the hexadecane sample (FIG. 2A). With a moderate centrifugal force applied (500×g, 1 min), clear phase separation was found in all the samples (FIG. 2B). The amount of residual oil in the subnatant layer was examined under an optical microscope. Interestingly, despite having the fastest separation, the hexane sample had the most remaining oil droplets (FIG. 2C). There was very little oil remaining in the decane sample (FIG. 2D), where the major component of the subnatant was cell debris. Complete phase inversion from O/W to W/O was found in hexadecane sample (FIG. 2E), where water droplets containing cell debris were free-flowing in the hexadecane phase. These results suggested that the longer chain hydrocarbons can destabilise the algae biomass emulsions at a microscopic level, leading to a greater extent of phase inversion at a lower oil-biomass ratio. However, the extra energy required in the separation step due to the increased viscosity of the oil phase and the decrease in density difference between water and water-immiscible phase needs to be considered.

Effect of Oil-Biomass Ratio

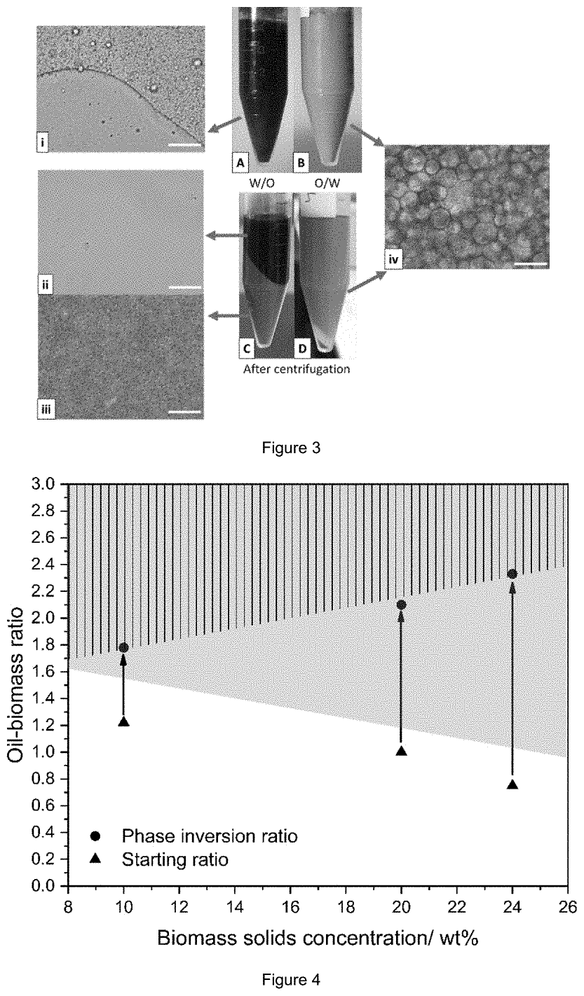

Altering the fraction of the water-immiscible phase can lead to catastrophic phase inversion. Based on the results from Example 7, the recovery process could be improved using an increasingly hydrophobic water-immiscible component. In the case of algal lipid recovery, the recovered algal lipids could be potentially used as the water-immiscible phase since the main component of the algal lipids are triglycerides, which are highly hydrophobic. In addition, by recycling the recovered lipids, no toxic solvents are needed for further recovery. This improves the quality of both the water and water-immiscible fractions and significantly reduces the energy required by avoiding the need for thermal removal of a conventional solvent (e.g. hexane). Due to the limitation of the resource of algal lipids, canola oil was used as a mimic of the recycled algal lipids. Ruptured biomass at a solids concentration of 20 wt % (pH=6.2) was used, with canola oil added to reach an oil-biomass ratio between 1.0:1.0 and 3.0:1.0 (v/v). Mixtures of canola oil and biomass at different oil-biomass ratios were hand mixed before application of ultrasound at a power density of 3.2 W/mL for 10 s. The emulsions in the samples were examined both visually and by optical microscopy. Sonication of the mixture made at an oil-biomass ratio of 1.5:1.0 resulted in phase inversion to produce a W/O emulsion. Sonication of the mixture made at an oil-biomass ratio of 1.0:1.0 did not result in phase inversion and remained as an O/W emulsion. The bulk appearance of the W/O and O/W oil-biomass emulsions were drastically different, with the W/O emulsions appearing very liquid in consistency and dark-green in colour due to the oil-soluble chlorophyll, whereas the O/W emulsions were pale-green in colour and with a highly viscous, gel-like consistency (FIGS. 3A and B). When centrifuged at 1000×g for 5 min, clear separation of the W/O samples occurred, whereas no visible water-oil phase separation could be observed in the O/W samples (note that the large pale-green upper layer in FIG. 3 D was the emulsion and the clear dark-green bottom layer was the separated water). The emulsion layers were inspected by optical microscopic images (FIG. 3 i-iv). A clear oil phase (FIG. 3ii) and an oil-free biomass layer (FIG. 3iii) were achieved after centrifugation of the W/O emulsion produced at an oil-biomass ratio of 1.5:1.0. In comparison, oil droplets stabilised by the viscous biomass matrix was found even after centrifugation of the O/W emulsion produced at an oil-biomass ratio of 1.0:1.0. The minimum oil-biomass ratio to achieve shear-induced phase inversion was found to be 1.25:1.0 in this system. Similar experiments were performed using a high-pressure homogeniser as the shear-inducer, with the results confirming that HPH could phase invert this emulsion type at this oil-biomass ratio, similarly to the ultrasonic system.

HPH Pressure and Number of Processing Passes

The preliminary tests described above were conducted using ultrasonication as the shearing method for phase inversion. It was found that increasing sonication power density increased the recovery of algal lipids. The effect of HPH pressure was tested at 30, 60 and 100 MPa, using a mixture of canola oil and biomass (20 wt %, pH=6.2) at an oil-biomass ratio of 3.0:1.0 (v/v). The effect of using multiple processing passes (1, 3 or 6 was also examined at an applied pressure of 30 MPa using the same oil-biomass ratio mixture. After passing through the HPH the desired number of times, the water-in-oil emulsions were subjected to centrifugation at 1500×g for 5 min.

The extraction and separation efficiencies resulting from different HPH operating conditions can be found in Table 1. For a single pass, the extraction efficiency was found to double from 36% to 73% when the applied pressure was increased from 30 MPa to 60 MPa. In addition, the separation efficiency also increased from 78% to 95%. The low recovery efficiency at 30 MPa compared to 60 MPa could be due to incomplete phase inversion due to insufficiently high shear to fully overcome the viscous aqueous biomass barrier to release the trapped lipid droplets. However, no significant increase in extraction efficiency was found with a further pressure increase from 60 MPa to 100 MPa, indicating the potential to find optimum operating pressures for energy-efficient extraction. Extraction efficiency was also increased from 36% to 65% with an increase in the number of processing passes from 1 to 3 at an applied pressure of 30 MPa. A further slight increase in extraction was observed with 6 passes.

Comparing the results obtained at different HPH pressures and number of passes, it can be seen that increasing the applied pressure had a greater impact on the recovery efficiency than increasing the number of passes. Additionally, it can be seen that the phase inversion on a microscopic scale could be achieved at relatively low pressures (less than 100 MPa), which would be favourable for energy efficiency.

To highlight the importance of the high-shear environment created by ultrasonication and HPH, the extraction of algae lipids was benchmarked against a lower-shear form of agitation, in which the same oil-biomass mixture was subjected to 72-h of bulk rotation on a rotation station at 10 rpm. The resulting extraction efficiency was found to be less than 10%. The localised high-intensity shear environment produced during ultrasonication and HPH is able to deform the highly elastic biomass matrix with ease at a microscopic-scale, producing high interfacial areas and strong forces for coalescence allowing a significantly improved mass transfer. The use of high-shear phase inversion to produce microscopic W/O emulsions, enabled the lipids inside the highly stable biomass matrix to be released and coalesced into the continuous (water-immiscible) phase.

TABLE 1
The extraction and separation efficiency of algal lipids processed
under different HPH operating conditions using ruptured biomass
with a solids concentration of 20 wt %, pH = 6.2.

| HPH operating | Extraction | Separation |
|---|---|---|
| conditions | efficiency (%) | efficiency (%) |
| 30 MPa 1 pass | 36 | 78 |
| 30 MPa 3 pass | 65 | 97 |
| 30 MPa 6 pass | 68 | 91 |
| 60 MPa 1 pass | 73 | 95 |
| 100 MPa 1 pass | 72 | 94 |

Effects of Solids Concentration

The solids concentration was found to have a great impact on the recovery of algal lipids mainly due to a change in interfacial activity, which can be related to the following three effects: 1) the water content; 2) the concentration of interfacially active components, such as proteins, polysaccharides and cell debris; 3) the hysteresis of phase inversion due to the emulsion history. Since high-shear methods such as ultrasonication and high-pressure homogenisation can potentially create highly stable emulsions, it is therefore important to produce the desired emulsion type. In the case of algal lipid recovery, the importance of maintaining an oil-continuous system can be seen in Table 2, where the rheological behaviour of the ruptured biomass (aqueous) and oil-continuous biomass mixture at a solids concentration of 5, 10 and 20 is shown. The viscosity of the biomass increased drastically from 172.4 cP to 16250 cP when solids concentration increased from 5 to 20 wt %, resulting in a semi-solid/gel-like material, in which the lipids are well-stabilised by an abundance of interfacially active components, such as proteins, polysaccharides and cell debris. Due to the presence of these interfacial-active components, any extracting solvents that are added can also be emulsified into the aqueous biomass matrix, even under low-shear agitation, thereby increasing the difficulty of phase separation. In comparison, by introducing the canola oil (60 cP, 25° C., 1 s⁻¹) to produce an oil-continuous W/O emulsion, the viscosity of the mixture was reduced 9 fold at 10 wt %, and 37-fold at 20 wt % at a shear rate of 1 s⁻¹. These results demonstrate the dramatic viscosity reduction that can be achieved by producing a water-immiscible (e.g., canola oil) continuous phase compared to an aqueous biomass, which can be of benefit throughout the recovery process and which is particularly important at a higher solids concentration.

TABLE 2
Viscosity of an aqueous slurry of ruptured biomass
and an oil-continuous oil-biomass mixture (oil-to-
biomass ratio of 3.0:1.0) at different shear rates.

| Sample | Biomass solids | |
|---|---|---|
| (continuous | concentration | Viscosity (cP, 25° C.) |

| phase) | (wt %) | 1 s⁻¹ | 50 s⁻¹ |
|---|---|---|---|
| Biomass | 5 | 172.4 | 6.71 |
| (Water) | 10 | 3629 | 62.09 |
| | 20 | 16250 | 1638 |
| Oil-biomass | 5 | 243.8 | 54.17 |
| mixture | 10 | 394.2 | 62.83 |
| (Canola oil) | 20 | 436.6 | 168.6 |

Further experiments were conducted to demonstrate the importance of producing an oil-continuous emulsion through controlling the oil:biomass ratio (v/v) and the provision of high-shear. It is known that the decrease of oil-to-water ratio could lead to phase inversion from W/O to O/W. In the case of algal lipids recovery, the O/W emulsions are typically gel-like and highly stable, which are unfavoured. Therefore, the minimum threshold oil-to-biomass ratio where the W/O formation can be formed after the high-shear process is one of the key parameters required. To explore the effect of solid concentration on the emulsion formation during the high-shear processing, ruptured algal biomass was made up to solids concentrations of 10, 20 and 24 wt % at an unadjusted pH of 6.2 using Milli-Q water as a diluent. The minimum oil-to-biomass ratio to produce a W/O emulsion under high-shear environment was tested at each solids using ultrasonication at 3.2 W/mL for 30 s using the procedure described in Example 8.

Table 3 shows the minimum oil-to-biomass ratio that allowed phase inversion to produce an O/W emulsion at different solids concentrations. The minimum oil-to-biomass ratio (v/v) decreased from 1.5:1.0 to 1.0:1.0 with an increase in the solids concentration from 10 to 24 wt %. The reduction in this ratio could be due to the reduced water content at higher solids concentration, which could restrict the interfacial activity of all the surface-active components present in the aqueous phase. Consistent with this, microscopic observations indicated an increase in the thickness of the films at the water-oil interface as the solids concentration decreased.

TABLE 3
The minimum canola oil-to-biomass ratio required
to produce an W/O emulsion as a function of
solids concentration of biomass (pH = 6).

| Biomass solids | Minimum oil-biomass |
|---|---|
| concentration (wt %) | ratio (v/v) |
| 10 | 1.5 |
| 20 | 1.25 |
| 24 | 1.0 |

Additional experiments were conducted to demonstrate the importance of controlling the proportions of the aqueous biomass and the water-immiscible component in order to achieve an oil-continuous phase. Canola oil was introduced to the biomass to produce mixtures with less the minimum oil-to-biomass ratio (v/v) at the respective solids concentrations (1.22:1.0 at 10 wt %, 1.0:1.0 at 20 wt % and 0.75:1.0 at 24 wt %, respectively). The mixtures were subjected to the same ultrasonication process. However, rather than phase inverting when subjected to high shear, highly stable O/W emulsion gels were produced (Example 8, FIG. 3B). Subsequently, more canola oil was added to these 0/W biomass emulsion gels, in order to reach the minimum oil-biomass ratio in Table 3. The samples were then subjected to ultrasonication following the same process. For all the samples, phase inversion did not occur. The process of oil addition and ultrasonication processing was repeated with further incremental increases in the oil content, until phase inversion was achieved. FIG. 4 presented the final oil-to-biomass ratios that were required when the starting oil-to-biomass ratio was below the determined minimum ratio. The final ratio was higher than the minimum ratio, and the amount of additional oil required increased with increasing solids concentration. The extra oil addition could be attributed to the hysteresis of phase inversion due to the high stability of the oil-in-water emulsion type.

Based on the understanding developed above, the recovery efficiency was then determined for HPH processed emulsions at different solids concentrations and oil-to-biomass ratios using the same analysis protocol described in Example 9. HPH processing was performed using a single pass at 60 MPa. Despite higher oil-to-biomass ratios used for the low solids concentrations (5 and 9 wt %), the extraction rate was only around 50%, which could be because oil-in-water emulsification was also promoted by high-shear when there was excessive water content. Extraction efficiency was improved at higher solids concentrations, from 73% at 20% solids to 80% at 23% solids. However, a slightly lower separation efficiency indicates that centrifugal separation may instead become the limiting factor at excessively high solids concentrations.

TABLE 4
The extraction and separation efficiency of algal lipids processed
using a single pass HPH at 60 MPa at different solids concentrations
of ruptured biomass at their natural pH.

| Biomass solids | Extraction | Separation | Oil-biomass |
|---|---|---|---|
| concentration (wt %) | efficiency (%) | efficiency (%) | ratio (v/v) |
| 5 | 49 | 81 | 2.0 |
| 9 | 52 | 85 | 2.0 |
| 20 | 73 | 95 | 1.5 |
| 23 | 80 | 93 | 1.5 |

Effect of pH of Biomass on Recovery Efficiency

pH is one of the key parameters that alters the interfacial activity of a surface-active component. In a complex emulsion system, such as algal biomass where multiple surface-active components (e.g. proteins and polysaccharides) exist, variation of pH could lead to solubility and structural alteration of components, resulting in both interfacial activity and viscosity change. It was found that the pH of the algae biomass decreased from 9 to 6 following incubation. The drop in pH could be due to the acidification of CO₂ created by the metabolism of algae cells and the release of cytoplasmic material and components (protein and polysaccharides) from the cell walls. After the cell rupture step, the pH of the biomass was found to be 6.2.

The effect of pH was determined using ruptured biomass with a solids concentration of 23 wt %, with the pH of the biomass increased to 8.8 and 12 using thoroughly mixed sodium hydroxide powder. Table 5 shows that the viscosity of the biomass approximately halved when the pH was increased from 6.2 to 8.8. Further pH elevation led to a slight viscosity increase from 27570 to 33860 cP.

TABLE 5
Viscosity of ruptured biomass at different
pH, at a shear rate of 1 s⁻¹.

| pH of ruptured biomass | Viscosity (cP) |
|---|---|
| 6.2 | 63930 |
| 8.8 | 27570 |
| 12 | 33860 |

To avoid the saponification of lipids at high pH, the recovery efficiency was tested between the samples at pH=6.2 and pH=8.8 (Table 6). The extraction efficiency was found to increase from 80% to 94% according to analysis by UV-vis of the recovered water-immiscible phase and verified by Bligh and Dyer extraction of the residual biomass. The slight increase in separation efficiency could be due to the viscosity reduction by the pH adjustment.

TABLE 6
The extraction and separation efficiency of algal lipids at
pH = 6.2 and pH = 8.8 at a solids concentration of
23 wt % using an oil-biomass ratio of 1.5, single HPH pass at 60 MPa.

| pH of rupture | Extraction | Separation |
|---|---|---|
| biomass | efficiency(%) | efficiency(%) |
| 6 | 80 | 93 |
| 8.8 | 94 | 95 |

### Example 2: Astaxanthin/Carotenoid Recovery from Haematococcus pluvialis by Shear-Induced Phase Inversion Method

Haematococcus pluvialis was cultivated as described elsewhere (see for example E. G. Baroni, K. Y. Yap, P. A. Webley, P. J. Scales and G. J. Martin, Algal Research, 2019, 39, 101454), from which ketocarotenoid, antioxidant pigments were accumulated. The solids concentration of the harvested algae was measured, gravimetrically, to be ˜15 wt %.

The biomass was partially ruptured by high-intensity low-frequency (20 kHz) ultrasound for 10 min with the pulse mode (5 s on and 10 s off). The temperature of the biomass was controlled at around 25-30° C. during the ultrasonication process. The pH of the partially ruptured biomass was 4.5. The minimum threshold oil-biomass ratio (v/v) to trigger catastrophic phase inversion was examined with hexane at pH=4.5 and 12.

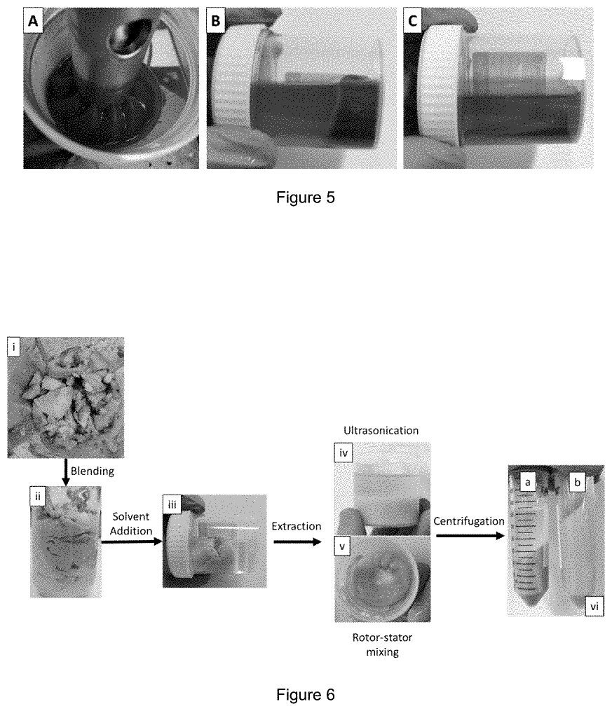

The recovery efficiency of shear-induced phase inversion method was determined at both pH levels (4.5 and 12) using hexane as the solvent. A pre-mixing of the biomass and hexane was conducted using a rotor-stator mixer at 12000 rpm (60 W) for 1 min using a pre-determined minimum hexane-biomass ratio at pH=4.5 and 12. The pre-mixed hexane-in-biomass emulsion was found to be highly stable and viscous (FIG. 5A) from which no observable phase separation could be achieved even after centrifugation at 500×g for 2 min. The shear-induced phase inversion was completed using high-intensity low-frequency ultrasound at 1 W/mL for 20 s, after which instant phase separation was observed. Noticeably, the dispersed biomass aqueous phase at pH=12 (FIG. 5C) was considerably less viscous compared to pH=4.5 (FIG. 5B), indicating a greater degree of phase inversion.

To highlight the significance of phase separation and natural sedimentation (due to the low viscosity of the solvent), the separated organic phase was directly decanted after 5 min of gravity settling, from which the decantable fraction (hexane removed under nitrogen flow) was determined. The biomass was then subjected to centrifugation at 5000×g for 5 min, after which another fraction of organic phase was collected, namely a centrifuged fraction. The total recovery per dry biomass weight was determined by adding the decantable and centrifuged fractions.

To obtain the total extractable content using hexane as the solvent, triplicate biomass samples were weighed and completely dried then subjected to a hexane extraction process at 55° C. for 48 h. The hexane fraction was collected, and the extracts were obtained by solvent evaporation at 55° C. under nitrogen gas flow. The total extractable per dry biomass weight was calculated using the weight of extractants over dry biomass, which was used for further quantitative comparison.

As shown in Table 7, a significant reduction of hexane-biomass ratio (v/v) was found at pH=12 from 3.5:1.0 to 2.5:1.0. In both cases, once the phase inversion has been successfully initiated, a high organic phase recovery could be readily achieved without centrifugation. However, the total recovery per dry biomass was found to be much higher at pH=12. The high recovery of organic compounds at high pH could be due to the lower emulsion stability, in line with the drastically lower hexane-biomass ratio. With the total extractable content (hexane as solvent) determined as ˜122 mg per gram dry biomass, the current extraction-separation method resulted in ˜75% and ˜21% recovery efficiency at pH 4.5 and 12, respectively.

TABLE 7
Minimum oil-biomass ratio, percentage of decantable organic fraction
after phase inversion over total collected volume, the mass concentration
of recoverable components and the final recovery efficiency at two
tested pH values, the solids concentration of 15 wt %.

| | Oil-biomass | Decantable/total | Total recovered per | Recovery |
|---|---|---|---|---|
| pH | ratio (v/v) | recovery (%) | dry biomass (mg/g) | (%) |
| 4.5 | 3.5 | 96 | 25 | 21 |
| 12 | 2.5 | 94 | 91 | 75 |

### Example 3: Recovery of Avocado Oil by Phase Inversion Separation Method

Fresh avocado fruits were sourced from a local market. Avocado pulps were obtained after peeling and destoning. The pulps were then pureed from which the solid content and total oil content (hexane extraction at 55° C. for 48 h) were determined to be ˜23 wt % and ˜11 wt %, respectively.

The minimum hexane-biomass ratio was determined at ˜23 wt % solid concentration as 1.0:1.0 (v/v). It is worth noting that the extraction of avocado oil cannot be performed by handshaking (FIG. 6 iii) due to the high viscosity of avocado biomass. Similar to other reported systems, the mixture became a stable and viscous biomass emulsion upon rotor-stator mixing, in which hexane ended up trapped inside the biomass matrix (FIG. 6 v). Shear-induced phase inversion was conducted by the same procedure described in example 12, after which rapid phase separation was observed after applying ultrasonication (FIG. 6 iv). In contrast, rotor-stator mixed emulsions were unbreakable after centrifugation (FIG. 6 vi, 100×g 1 min).

The avocado oil recovery was also tested at pH 5 and 13 along with its natural pH of 6.4. Recovery of avocado oil was determined with the same method as described in Example 12 (i.e., decantable and centrifuged fractions determined after hexane removal under nitrogen gas flow). The highest total recovery was ˜95% at natural pH after 20 s ultrasonication, where the majority was recovered from the decantable fraction after gravity settling. Both pH adjustments impaired the recovery, which was reduced to 81% (77% decantable) and 70% (67% decantable) at pH=5 and 13, respectively, consistent with the noticeable increase in viscosity of the biomass at pH 5 and 13.

An additional test was conducted using avocado puree that was aged for 7 days at 4° C. Oil recovery was reduced from 94% for fresh biomass to 80% for the aged biomass (pH=6.4), with a noticeable difference in biomass texture after phase inversion, from sandy particulates to sticky clusters. These changes could be related to the enzymatic reactions during the ageing process, and such reactions could be promoted at an elevated temperatures, such as in malaxation, the emulsion breaking step involved in cold press production (Da Silva, C., & Da Silva, C. (2018). U.S. Pat. No. 9,894,908. Washington, D.C.: U.S. Patent and Trademark Office).

Finally, it will be appreciated that various modifications and variations of the methods and compositions of the invention described herein would be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that is apparent to those skilled in the art are intended to be within the scope of the present invention.

1. A method of recovering organic components from an aqueous biomass, comprising:
(i) providing an aqueous biomass containing organic components;
(ii) treatment of the aqueous biomass to release intracellular organic components from within cells of the biomass to form a biomass suspension;
(iii) addition of a water-immiscible component to the biomass suspension to form a mixture comprising the biomass and water-immiscible component;
(iv) subjecting the mixture comprising biomass and water-immiscible component to high shear to form a water-in-water-immiscible-component emulsion; and
(v) separating the water-immiscible component phase containing the organic components from the water phase.

2. The method according to claim 1, wherein the organic components are selected from the group consisting of lipids, proteins, carbohydrates, pigments and combinations thereof.

3. The method according to claim 1, wherein the aqueous biomass that contains the organic components has a solids content of from 1% to 50% on a weight basis.

4. The method according to claim 1, wherein the biomass is an oleaginous biomass.

5. The method according to claim 1, wherein the biomass contains organisms selected from the group consisting of algae, plants (or parts thereof), fungi, bacteria, protists and combinations thereof.

6. The method according to claim 1, wherein the treatment comprises subjecting the biomass to a process selected from the group consisting of high-pressure homogenisation, bead milling, sonication, pulsed electric fields, osmotic stressing, enzymatic treatment, microwave irradiation, mechanical pressing or pureeing.

7. The method according to claim 6, the biomass is subjected to high-pressure homogenisation.

8. The method according to claim 6, the biomass is subjected to sonication.

9. The method according to claim 1, wherein the amount of the water-immiscible component added to the biomass suspension is sufficient to form a mixture with a ratio of water-immiscible component: biomass suspension of at least 1.0:1.0 (v/v).

10. The method according to claim 9, wherein the amount of water-immiscible component added to the biomass suspension is sufficient to form a mixture with ratio of water-immiscible component: biomass suspension of at least 1.5:1.0 (v/v).

11. The method according to claim 1, wherein the water-immiscible component is a water immiscible crude biomass extract.

12. The method according to claim 1, wherein the water-immiscible component is a water-immiscible solvent.

13. The method according to claim 1, wherein the mixture comprising biomass/water-immiscible component is subjected to high shear by sonication of the mixture.

14. The method according to claim 13, wherein the mixture comprising the biomass/water-immiscible component is subjected to sonication at a frequency of from 20 kHz to 200 kHz.

15. The method according to claim 13, wherein the mixture comprising the biomass and water-immiscible component is subjected to sonication at a frequency of from 20 kHz to 40 kHz.

16. The method according to claim 13, wherein the mixture comprising the biomass and water-immiscible component is sonicated at a power density of greater than 0.8 W/mL.

17. The method according to claim 1, wherein the mixture comprising the biomass and water-immiscible component is subjected to high shear by the subjection of the mixture comprising biomass and water-immiscible component to high-pressure homogenisation.

18. The method according to claim 17, wherein the high-pressure homogenisation is carried out at a pressure of from 10 MPa to 200 MPa.

19. The method according to claim 17, wherein the high-pressure homogenisation is carried out at a pressure of from 10 MPa to 150 MPa.

20. The method according to claim 17, wherein the high-pressure homogenisation is carried out at a pressure of from 30 MPa to 100 MPa.

21. The method according to claim 1, wherein separating the water-immiscible component phase from the water phase comprises centrifugation at a force less than 10,000×g for no more than 10 minutes.

22. The method according to claim 1, further comprising: (vi) isolating the organic components from the water-immiscible component phase.
